(19) United States
(12) Patent Application Publication
(10) Pub. No.: US 2023/0414460 A1
(43) Pub. Date: Dec. 28, 2023

(54) COMPOSITIONS COMPRISING CERAMIDE, POLYGLYCEROL CARBOXYLIC ACID ESTERS AND CHOLESTEROL

(71) Applicant: EVONIK OPERATIONS GmbH, Essen (DE)

(72) Inventors: Ursula MACZKIEWITZ, Essen (DE); Juergen Meyer, Essen (DE); Lisa Maus, Duisburg (DE); Gabriele Polak, Hagen (DE); Heike Blasko-Begoihn, Essen (DE); Jan Marian von Hof, Bochum (DE)

(73) Assignee: Evonik Operations GmbH, Essen (DE)

(21) Appl. No.: 18/248,224

(22) PCT Filed: Oct. 4, 2021
(86) PCT No.: PCT/EP2021/077250

§ 371 Date: Apr. 6, 2023

(30) Foreign Application Priority Data
Oct. 9, 2020 (EP) 20200973.4

(51) Int. Cl. A61K 8/06; A61K 8/37; A61K 8/63; A61K 8/68; A61Q 19/00

(52) U.S. Cl. CPC A61K 8/062; A61K 8/375; A61K 8/63; A61K 8/68; A61Q 19/00

(57) ABSTRACT

Compositions containing at least one ceramide, at least one polyglycerol carboxylic acid ester, and cholesterol in specific ratios by weight are provided.

### FIELD OF THE INVENTION

The invention relates to compositions comprising at least one ceramide, at least one polyglycerol carboxylic acid ester and cholesterol in specific ratios by weight.

### PRIOR ART

EP3117821 discloses a process for producing a ceramide dispersion composition in which a mixture of ceramide, non-ionic surfactant and polyhydric alcohol is heated to a temperature of equal to or higher than 100° C.

EP2295032 discloses ceramide dispersions comprising natural ceramide and at least one surfactant, wherein the ceramide dispersions in the at least one surfactant comprise at least one polyglycerol fatty acid ester having an HLB of 10 to 16.

EP975325 discloses a composition for topical application, comprising a combination of a free sphingoid base and a ceramide.

U.S. Pat. No. 8,710,034 discloses compositions comprising ceramide and cholesterol for improving the barrier function of skin.

US2006198800A1 discloses a skin care composition comprising: a safe and effective amount of at least one anti-wrinkle agent and a safe and effective amount of a natural peeling complex.

The object of the invention was to provide ceramide-containing compositions which further improves the cosmetic properties of the ceramides present.

### DESCRIPTION OF THE INVENTION

It has been found that, surprisingly, the composition described in claim 1 is able to solve the problem addressed by the invention.

The present invention therefore relates to a composition comprising at least one ceramide, at least one polyglycerol carboxylic acid ester and cholesterol in specific ratios by weight.

The invention further relates to the use of the composition according to the invention for increasing the barrier function of skin.

An advantage of the present invention is that the compositions according to the invention have increased storage stability and are thus slower to change in their nature over time, particularly with regard to their viscosity, compared to ceramide-containing compositions according to the prior art. Another advantage of the present invention is that the compositions according to the invention tolerate a higher number of freeze-thaw steps without significant loss of viscosity, compared to ceramide-containing compositions according to the prior art.

One advantage of the composition according to the invention is that the composition in formulations has superior sensory properties which lead to an improved skin feel and/or hair feel.

It is a further advantage of the composition according to the invention that the composition in formulations stimulates in vitro human follicular dermal papilla cells (HFDPCs) to proliferate, which in vivo equates to stimulation of hair growth.

It is a further advantage of the composition according to the invention that the composition in formulations has improved distributability compared to the individual components.

It is a further advantage of the composition according to the invention that the composition in formulations has improved absorption compared to the individual components.

It is a further advantage of the composition according to the invention that the composition in formulations has reduced oiliness compared to the individual components.

It is a further advantage of the composition according to the invention that the composition in formulations has reduced waxiness compared to the individual components.

It is a further advantage of the composition according to the invention that the composition in formulations has improved glidability compared to the individual components.

It is a further advantage of the composition according to the invention that the composition in formulations has reduced tackiness compared to the individual components.

It is a further advantage of the composition according to the invention that the composition in formulations has improved silkiness/velvetiness compared to the individual components.

A further advantage of the composition according to the invention is that the composition improves skin roughness and skin smoothness more potently than ceramide-containing compositions according to the prior art.

A further advantage of the composition according to the invention is that they regenerate a barrier damaged by SDS more strongly than ceramide-containing compositions according to the prior art.

In the context of the present invention, the term “ceramide” is understood to mean acylated sphingoid bases, where the sphingoid bases are preferably selected from sphingosine, sphinganine, 6-hydroxysphingosine and phytosphingosine.

The “pH” in connection with the present invention is defined as the value which is measured for the relevant composition at 22° C. after stirring for five minutes using a pH electrode calibrated in accordance with ISO 4319 (1977).

The polyglycerol carboxylic acid esters present according to the invention are mixtures of different substances; it is therefore clear to those skilled in the art that the numeric values specified are average values for the mixture.

In the context of the present invention, the term “polyglycerol” is to be understood as meaning a polyglycerol which may also comprise glycerol. Consequently, for the purposes of calculating amounts, masses and the like, any glycerol fraction should also be taken into consideration. Owing to its polymeric property, the polyglycerol is a statistical mixture of various compounds. Polyglycerol may have ether bonds formed between two primary, one primary and one secondary or else two secondary positions of the glycerol monomers. For this reason, the polyglycerol base skeleton does not usually consist exclusively of linearly linked glycerol units, but may also comprise branches and rings. For details see, for example, “Original synthesis of linear, branched and cyclic oligoglycerol standards”, Cassel et al., J. Org. Chem. 2001, 875-896.

The same applies to the term “polyglycerol carboxylic acid ester” in connection with the present invention.

Unless stated otherwise, all percentages (%) given are percentages by mass.

The present invention therefore relates to a composition comprising
A) at least one ceramide,
B) at least one polyglycerol carboxylic acid ester and
C) cholesterol and/or at least one cholesterol derivative selected from the group comprising cholesterol sulfate, particularly the potassium salt of cholesterol sulfate, potassium cholesteryl sulfate, cholesterol hydrogen succinate and 7-dehydrocholesterol, preferably in a total amount of 0.1% by weight to 3.0% by weight, preferably 0.1% by weight to 2.0% by weight, particularly preferably 0.2% by weight to 1.0% by weight, where the percentages by weight refer to the total composition,
   characterized in that
   the ratio by weight of all ceramides present to the sum of cholesterol present and/or of the at least one cholesterol derivative is from 1:0.1 to 1:0.9, preferably 1:0.1 to 1:0.8, particularly preferably 1:0.2 to 1:0.7.

A preferred composition according to the invention is characterized in that the ratio by weight of all ceramides present to all polyglycerol carboxylic acid esters present is from 1:1 to 1:30, preferably from 1:1 to 1:25, particularly preferably from 1:2 to 1:20.

It is preferred in accordance with the invention that the composition according to the invention is characterized in that it comprises
D) at least one sphingoid base, preferably selected from the group comprising sphingosine, sphinganine, 6-hydroxysphingosine, N-acetylphytosphingosine and phytosphingosine, especially phytosphingosine.

Component D) is preferably present according to the invention in an amount of 0.01% by weight to 2.0% by weight, preferably 0.02% by weight to 1% by weight, particularly preferably 0.02% by weight to 0.8% by weight, based on the total composition according to the invention.

According to the invention, the ratio by weight of component A) to component D) in the composition according to the invention is preferably from 1:1 to 1:0.01, preferably from 1:0.8 to 1:0.02, particularly preferably from 1:0.7 to 1:0.05.

It is preferred in accordance with the invention that the composition according to the invention is characterized in that it comprises
E) at least one free fatty acid, preferably selected from the group of fatty acids having a chain length of 12 to 40, preferably 14 to 24, particularly preferably 16 to 22 carbon atoms. Component E) is preferably present according to the invention in an amount of 0.01% by weight to 3.0% by weight, preferably 0.05% by weight to 2.0% by weight, particularly preferably 0.1% by weight to 1.0% by weight, based on the total composition according to the invention.

According to the invention, the ratio by weight of component B) to component E) in the composition according to the invention is preferably from 1:0.001 to 1:1, preferably from 1:0.01 to 1:0.5, particularly preferably from 1:0.02 to 1:0.1.

It is preferred in accordance with the invention that the composition according to the invention is characterized in that said composition comprises 20% by weight to 99% by weight, preferably 40% by weight to 97% by weight, particularly preferably 50% by weight to 95% by weight water, based on the total composition.

enthält.

Preferred compositions according to the invention are active ingredient concentrates having a high active content; a preferred composition according to the invention of this kind is characterized in that said composition comprises component A), B) and C) in a total amount of 1.0% by weight to 25% by weight, preferably 2.0% by weight to 20% by weight, particularly preferably 4.0% by weight 35 to 15% by weight, based on the total composition.

Compositions preferred in accordance with the invention comprise at least two ceramides, preferably at least three ceramides, particularly preferably precisely three ceramides.

Preferably present in the composition according to the invention are ceramides selected from the group comprising ceramide NP, ceramide AP, ceramide EOP, ceramide NDS, ceramide ADS, ceramide EODS, ceramide NS, ceramide AS, ceramide EOS, ceramide NH, ceramide AH and ceramide EOH, preferably selected from the group comprising ceramide NP, ceramide AP, ceramide NS, ceramide EOP and ceramide EOS.

Compositions preferred in accordance with the invention are characterized in that the polyglycerol carboxylic acid ester of component B is composed of a polycerol having a degree of polymerization of 2.0 to 25, preferably of 2.5 to 20, particularly preferably of 3.0 to 15.

The degree of polymerization of the polyglycerol N is calculated via its hydroxyl number (OHN, in mg KOH/g) according to the following formula:

$$N = \frac{( 112200 - 18 \cdot OHV )}{( 74 \cdot OHV - 56100 )}$$

Suitable methods for determining the hydroxyl number are particularly those according to DGF C-V 17 a (53), Ph. Eur. 2.5.3 Method A and DIN 53240.

The composition of the present invention is preferably characterized in that the polyglycerol carboxylic acid ester of component B) is composed of at least one carboxylic acid selected from fatty acids, in particular having 12 to 26, preferably 14 to 24, particularly preferably 16 to 22 carbon atoms.

The composition of the present invention is preferably characterized in that the polyglycerol carboxylic acid ester of component B) does not contain any dicarboxylic, preferably any polycarboxylic, acid esterified into the polyglycerol carboxylic acid ester.

The fatty acids are generally unbranched and consist of an even number of carbon atoms. Any double bonds have cis configuration. Examples of fatty acids are: caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, linolenic acid, petroselic acid, elaidic acid, arachic acid, behenic acid, erucic acid, gadoleic acid, linolenic acid, eicosapentaenoic acid, docosahexaenoic acid and arachidonic acid.

The at least one carboxylic acid from which the polyglycerol carboxylic acid ester of component B) is composed according to the invention is particularly preferably selected from palmitic acid, stearic acid and behenic acid.

It is particularly preferred in accordance with the invention that the polyglycerol carboxylic acid ester of component B) is composed of mixtures of fatty acids, especially mixtures of at least two selected from palmitic acid, stearic acid and behenic acid, wherein the sum of these makes up at least 60% by weight, preferably at least 75% by weight, even more preferably at least at least 85% by weight, based on all fatty acids present in the mixture. In this context, it is particularly preferred in accordance with the invention if the ratio by weight of palmitic acid to stearic acid to behenic acid is from 1.0:3.0 to 1.0:3.0 to 1.0, preferably from 1.2:2.1 to 1.4:2.4 to 1.0.

The polyglycerol carboxylic acid ester present in component B) according to the invention can be provided in accordance with the invention by mixing different separately obtained polyglycerol carboxylic acid esters. Thus, by way of example, by mixing a polyglycerol palmitate with a polycerol behenate.

The polyglycerol carboxylic acid ester present in component B) according to the invention is characterized preferably in accordance with the invention in that it has a saponification number of to 199 mg KOH/g, preferably 30 to 100 mg KOH/g, particularly preferably 40 to 70 mg KOH/g. By this, an extraordinary dispersion stability is assured.

The determination of the saponification number is carried out by those skilled in the art according to DGF C-V 3 or DIN EN ISO 3681.

Preferred compositions according to the invention are particularly formulations, especially in the form of a cosmetic, pharmaceutical or dermatological formulation.

From the aforementioned active ingredient concentrates, formulations can be produced by mixing in which the active ingredient concentration is reduced compared to the aforementioned concentrates; such an alternative, preferred composition according to the invention is characterized in that it comprises component A), B) and C) in a total amount of 0.01% by weight to 1.25% by weight, preferably 0.01% by weight to 1.00% by weight, particularly preferably 0.02% by weight to 1.00% by weight, based on the total composition, and is particularly preferably a cosmetic, pharmaceutical or dermatological formulation.

The formulations according to the invention can further comprise at least one additional component selected from the group of

emollients,
emulsifiers,
thickeners/viscosity regulators/stabilizers,
UV light protection filters,
antioxidants,
hydrotropes (or polyols),
solids and fillers,
film formers,
pearlescence additives,
deodorant and antiperspirant active ingredients,
insect repellents,
self-tanning agents,
preservatives,
conditioning agents,
perfumes,
dyes,
odour absorbers,
cosmetic active ingredients,
care additives,
superfatting agents,
solvents.

Substances which can be used as exemplary representatives of the individual groups are known to those skilled in the art and can be found for example in German application DE 102008001788.4. This patent application is hereby incorporated as reference and thus forms part of the disclosure.

As regards further optional components and the amounts used of these components, reference is made expressly to the relevant handbooks known to those skilled in the art, for example K.

Schrader, “Grundlagen and Rezepturen der Kosmetika [Cosmetics—fundamentals and formulations]”, 2nd edition, pages 329 to 341, Hüthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

Compositions of the present invention which are preferred according to the invention are emulsions, in particular oil-in-water emulsions.

A particularly preferred composition according to the invention is characterized in that said composition has a pH in the range of 4.0 to 8.0, preferably 4.5 to 7.4 particularly preferably 5.0 to 7.2.

The present invention further relates to the use of a composition according to the invention for increasing the barrier function of skin.

The examples listed below describe the present invention by way of example, without any intention that the invention, the scope of application of which is apparent from the entirety of the description and the claims, be restricted to the embodiments specified in the examples.

The following figures form part of the examples:

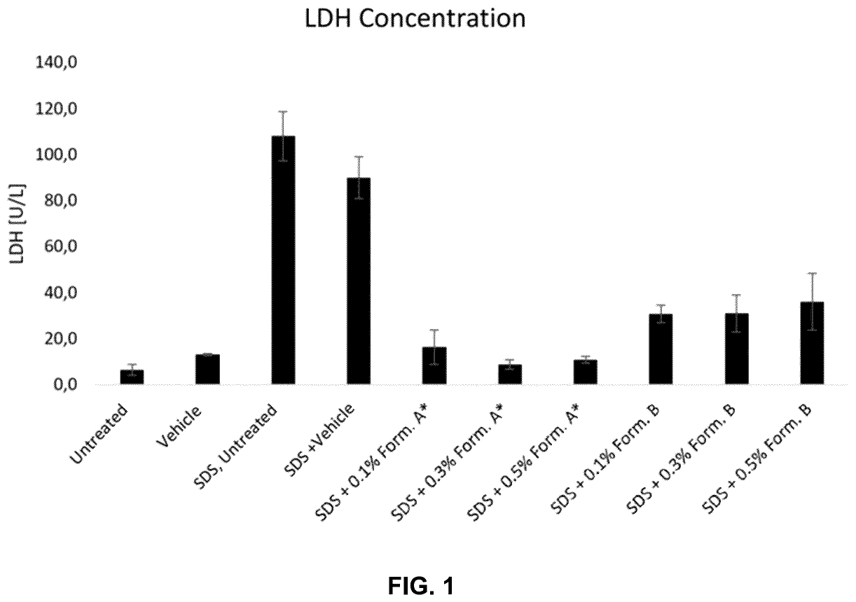

FIG. 1: LDH concentration in the supernatant of the skin models, measured 24 h after application of the test formulations to the skin models previously damaged with SDS.

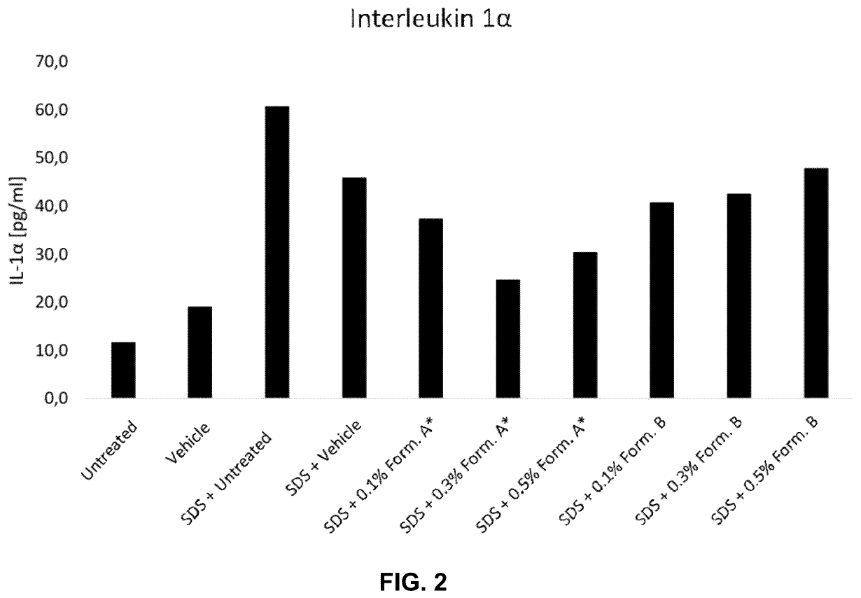

FIG. 2: Interleukin 1α concentration in the supernatant of the skin models, measured 24 h after application of the test formulations to the skin models previously damaged with SDS.

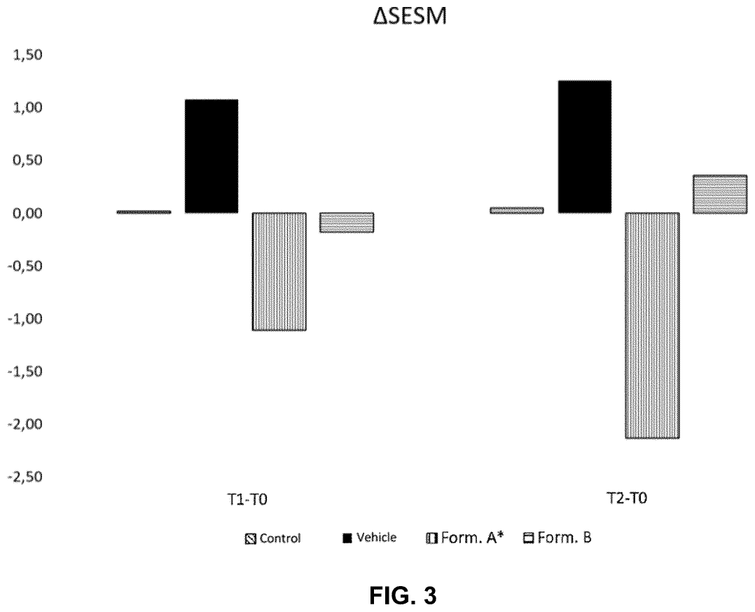

FIG. 3: The diagram shows the difference of the SEsm parameter to the starting value TO. A decrease in SEsm is equivalent to an improvement in skin smoothness.

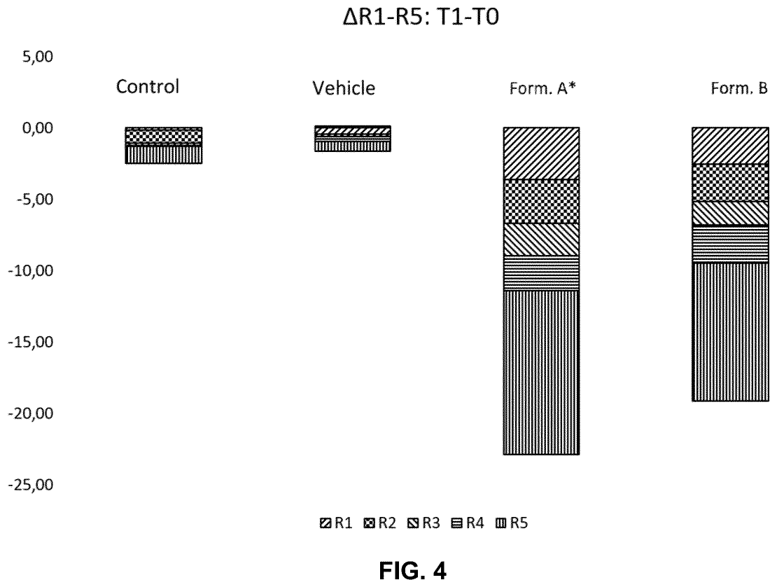

FIG. 4: The diagram shows the difference of the roughness parameter to the starting value TO after one week's application of the test formulations.

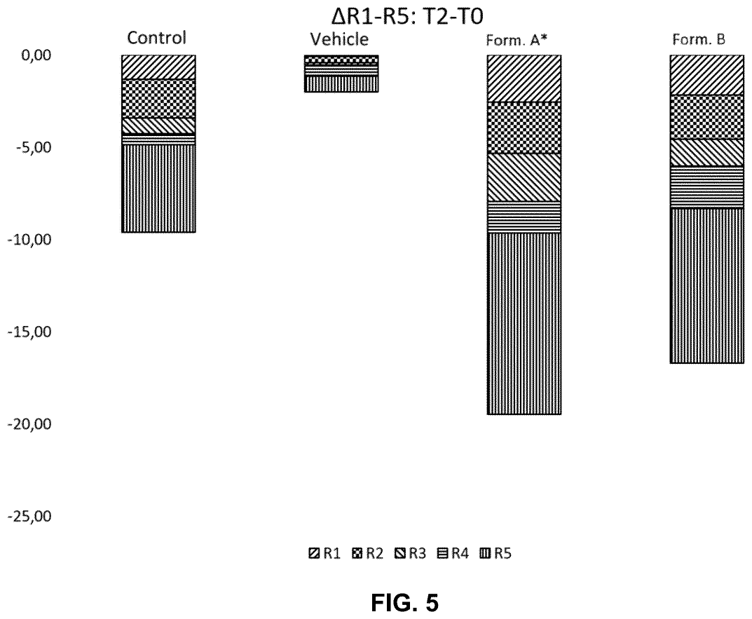

FIG. 5: The diagram shows the difference of roughness parameter to the starting value TO after two weeks' application of the test formulations.

### EXAMPLES

Inventive examples are marked with an *.

### Example 1: Synthesis of the Polyglycerol Carboxylic Acid Esters Used (Polyglycerol-6 Esterified with C16-, C18- and C22-Carboxylic Acids)

A mixture of commercially available polyglycerol-6 (Spiga Nord S.p.A.; 245.0 g, hydroxyl number=980 mg KOH/g), a technical grade mixture of stearic acid and palmitic acid (79.0 g, acid number=207 mg KOH/g, C16/C18:0˜47:53), behenic acid (28.3 g, acid number=165.5 mg KOH/g) and Na₂CO₃ (2.0 g) was heated to 240° C. over 3 h with introduction of nitrogen and the mixture was then stirred at this temperature and the resulting water removed continuously until an acid number of 1.0 had been reached. The resulting product had a saponification number of 61 mg KOH/g.

### Example 1b: Synthesis of the Polyglycerol Carboxylic Acid Esters Used (Polyglycerol-6 Esterified with C16- and C18-Carboxylic Acids)

A mixture of commercially available polyglycerol-6 (Spiga Nord S.p.A.; 223.7 g, hydroxyl number=980 mg KOH/g), a technical grade mixture of stearic acid and palmitic acid (79.0 g, acid number=207 mg KOH/g, C16/C18:0˜47:53) and Ca(OH)₂ (15 mg) was heated to 240° C. over 3 h with introduction of nitrogen and the mixture was then stirred at this temperature and the resulting water removed continuously until an acid number of 1.0 had been reached. The resulting product had a saponification number of 69 mg KOH/g.

### Example 1c: Synthesis of the Polyglycerol Carboxylic Acid Esters Used (Polyglycerol-10 Esterified with C16- and C18-Carboxylic Acids)

A mixture of commercially available polyglycerol-10 (Spiga Nord S.p.A.; 283.9 g, hydroxyl number=898 mg KOH/g), a technical grade mixture of stearic acid and palmitic acid (79.0 g, acid number=207 mg KOH/g, C16/C18:0˜47:53), behenic acid (28.3 g, acid number=165.5 mg KOH/g) and Na₂CO₃ (2.7 g) was heated to 240° C. over 3 h with introduction of nitrogen and the mixture was then stirred at this temperature and the resulting water removed continuously until an acid number of 1.0 had been reached. The resulting product had a saponification number of 58 mg KOH/g.

### Example 1d: Synthesis of the Polyglycerol Carboxylic Acid Esters Used (Polyglycerol-10 Esterified with C8-, C10-, C16- and C18-Carboxylic Acids)

A mixture of commercially available polyglycerol-10 (Spiga Nord S.p.A.; 310.0 g, hydroxyl number=898 mg KOH/g), a technical grade mixture of stearic acid and palmitic acid (79.0 g, acid number=207 mg KOH/g, C16/C18:0-47:53), behenic acid (28.3 g, acid number=165.5 mg KOH/g), a technical grade mixture of caprylic acid and capric acid (5.00 g, acid number=362 mg KOH/g, C8/C10˜58:42) and Na₂CO₃ (32.0 g) was heated to 240° C. over 3 h with introduction of nitrogen and the mixture was then stirred at this temperature and the resulting water removed continuously until an acid number of 1.0 had been reached. The resulting product had a saponification number of 57 mg KOH/g.

### Example 2: Regeneration of Damaged Skin Barrier

The following formulations are used; Example A is inventive, Example B corresponds to the prior art as described in Example 9 of US2006198800.

| | Example A (w/w %)* | Example B (w/w %) |
|---|---|---|
| Polyglycerol from | 6.0 | |
| Example 1 | | |
| Sodium Lauroyl Lactylate | | 10 |
| Ceramide EOP | 0.001 | 0.001 |
| Ceramide NP | 1 | 1 |
| Ceramide AP | 0.5 | 0.5 |
| Cholesterol | 0.5 | 0.5 |
| Phytosphingosine | 0.5 | 0.5 |
| Behenic acid | 0.5 | |
| Preservative | q.s. | q.s |
| Water | to 100.0 | to 100.0 |

The test described below is carried out on epidermis skin models (EpiCS, SkinInVitro, Troisdorf). These three-dimensional skin models consist of epidermis, proliferating keratinocytes and a stratum corneum having an intact barrier function. In order to investigate the regenerating effect of the test formulations on a damaged skin barrier, the skin models are damaged with 0.325% aqueous sodium dodecyl sulfate solution (SDS) for 40 minutes. The test formulations are then applied. The test formulations used are Example A* and Example B at a concentration of 0.1, 0.3 and 0.5% in water. A vehicle is also tested. This is Ultrapure water. The application duration is 1 hour.

There is a change of media after application. The supernatants are removed on the following day. These supernatants are used for quantifying the enzyme lactate dehydrogenase (LDH) and interleukin 1α. The lactate dehydrogenase enzyme is released, inter alia, on cell membrane damage. Interleukin 1α is a protein which is expressed by cells during inflammatory processes. FIG. 1 shoes the LDH concentration 24 hours after application of the test formulations to the skin models previously damaged with SDS. The damage to the skin barrier by sodium dodecyl sulfate sharply increases the concentration of LDH enzyme. This increase is reduced by both test formulations. The vehicle does not show this effect (SDS+vehicle). It is very clearly seen that the reduction by inventive Example A is more pronounced than that by prior art Example B.

FIG. 2 shoes the concentration of interleukin 1α (IL-1α) 24 hours after application of the test formulations to the skin models previously damaged with SDS. An inflammatory process is set in motion by damage to the skin barrier with SDS, which is evidenced by the sharp increase in the concentration of IL-1a. The vehicle (Ultrapure water) cannot stop this inflammatory process (SDS+vehicle), whereas in the skin models which have been treated with both test formulations after SDS damage, the expression of the IL-1a inflammatory marker is significantly reduced. In Example A this goes back down to the level of the undamaged skin models (vehicle); in test formulation B the expression of IL-1a is also distinctly reduced but it is significantly higher than in Example A.

The skin model test described above shows that both test formulations are capable of repairing the damage to the skin barrier caused by sodium dodecyl sulfate, but inventive Example A here shows clear advantages over the prior art Example B.

### Example 3: In Vivo Study on Skin Dryness

In this study, 40 volunteers with dry skin participate. The volunteers receive either two test formulations which they have to apply to the left and right inside of the forearms, or they receive one test formulation which they have to apply to one forearm whereas the other forearm remains untreated (control). The test formulations and the control are distributed according to the random principle but so that all test formulations and the control are tested on 20 volunteers.

The volunteers are instructed to use only the test formulations during the study. Normal cleansing/showers are however permitted.

The test formulations are applied twice daily over a period of two weeks. Prior to the start of application and after one and after two weeks, images of the inner forearms are recorded. A Visioscan VC 98 camera from CK electronic GmbH is used for this purpose. This is a specific camera which records enlarged black and white images of the skin surface. By means of the grey scale distribution in the image, the roughness parameters R1, R2, R3, R4 and R5 and the SEsm parameter (skin smoothness) are then determined with the aid of an integrated software. This parameter is proportional to the width and shape of the wrinkles. A more detailed description for calculating this parameter is found in the manual of the Visioscan VC 98 camera, Version “VC 98 english Fire Wire 04/2005 DK”.

The SEsm parameter has to decrease if skin smoothness is to improve.

The roughness parameters R1, R2, R3, R4 and R5 originally come from the metal industry and are defined in the provisions of DIN 4762-4768 as Ra-Rz. In the Visioscan software, these results are expressed as an index (in grey scales) and are linked to the original Ra-Rz values. The parameters have the following meanings:

| Parameter | Designation | DIN designation |
|---|---|---|
| R1 | Roughness depth | Rt |
| R2 | Maximum roughness depth | Rmax |
| R3 | Average roughness depth | Rz |
| R4 | Smoothness depth | Rp |
| R5 | Arithmetic mean roughness | Ra |

In order to determine the effectiveness of the test formulations, for each volunteer the difference in the parameters after one and two weeks from the starting value is determined (T1-T0, T2-T0) and the average value for the 20 volunteers calculated.

The following table shows the composition of the test formulation. This is an O/W lotion based on commercially available cosmetic raw materials. The following steps are required to prepare the formulation:
1. Phase A and B are heated to 70° C.
2. Phase B is added to phase A without stirring.
3. The mixture is then homogenized.
4. The emulsion is cooled to 60° C. with stirring and phase C is added.
5. The mixture is briefly homogenized once more.
6. The emulsion is further cooled to 30° C. with stirring. Below 40° C., phase D, E and F are added.

TABLE 1
Test formulations in vivo study

| Phase | Ingredient | Vehicle | Form. A* | Form. B |
|---|---|---|---|---|
| A | AXOL ® C 62 Pellets (Glyceryl Stearate Citrate) | 2.00 | 2.00 | 2.00 |
| | TEGO ® Alkanol 1618 (Cetearyl Alcohol) | 1.00 | 1.00 | 1.00 |
| | TEGOSOFT ® CT (Caprylic/Capric Triglyceride) | 4.50 | 4.50 | 4.50 |
| | TEGOSOFT ® TN (C12-15 Alkyl Benzoate) | 4.50 | 4.50 | 4.50 |
| B | Example A | | 5.00 | |
| | Example B | | | 5.00 |
| | Water | 85.20 | 80.20 | 80.20 |
| C | TEGO ® Carbomer 141 (Carbomer) | 0.20 | 0.20 | 0.20 |
| | TEGOSOFT ® CT (Caprylic/Capric Triglyceride) | 0.80 | 0.80 | 0.80 |
| D | Sodium Hydroxide (10%) | 0.70 | 0.70 | 0.70 |
| E | Verstatil ® PC (Phenoxyethanol; Caprylyl Glycol) | 1.00 | 1.00 | 1.00 |
| F | Perfume | 0.10 | 0.10 | 0.10 |
| | pH | 6.0-6.5 | 6.0-6.5 | 6.0-6.5 |

The result for skin smoothness is given in FIG. 3. The vehicle, i.e. the formulation base without active ingredient, is not able to improve skin smoothness, on the contrary there is deterioration. Only the test formulation comprising inventive Example A improves skin smoothness, whereas the test formulation with Example B has virtually no effect on skin smoothness.

In the case of the roughness parameters, both test formulations are capable of reducing skin roughness, whereas the vehicle shows no efficacy. Both after one week and after two weeks duration of application, formulation A according to the invention improves skin roughness more than formulation B of the prior art.

Summary of the study results: In summary, it can be shown on the basis of the results of the in vivo study that inventive Example A shows improved efficacy compared to Example B of the prior art with regard to improvement in skin smoothness and reduction of skin roughness in volunteers with dry skin.

### Further Formulations According to the Invention are as Follows:

| | | No. 4 | No. 5 | No. 6 | No. 7 | No. 8 | No. 9 | No. 10 |
|---|---|---|---|---|---|---|---|---|
| Phase | Example | w/w % | w/w % | w/w % | w/w % | w/w % | w/w % | w/w % |
| A | Polyglycerol from | 6.00 | 6.00 | 6.00 | 6.00 | 6.00 | 6.00 | 6.00 |
| | Example 1 | | | | | | | |
| A | Ceramide EOP | 0.10 | 1.45 | 0.10 | 0.16 | 0.10 | 0.99 | 0.11 |
| A | Ceramide EOS | | | | | | 0.33 | |
| A | Ceramide NP | 0.51 | | 0.51 | 0.52 | 0.54 | | 0.66 |
| A | Ceramide AP | 0.41 | | 0.41 | 0.10 | 0.45 | | |
| A | Ceramide NS | 0.18 | | 0.18 | 0.29 | 0.22 | | 0.21 |
| A | Cholesterol | 0.47 | 0.42 | 0.47 | 0.47 | 0.45 | 0.32 | 0.49 |
| A | Potassium Cholesterol | 0.11 | | 0.11 | 0.11 | | 0.08 | |
| | Sulfate | | | | | | | |
| A | N- | | | | 0.10 | | | |
| | Acetylphytosphingosine | | | | | | | |
| A | Phytosphingosine | | | | | | | 0.22 |
| A | Pentadecanoic acid | | | 0.03 | | | | |
| A | Heptadecanoic acid | | | 0.03 | | | | |
| A | Palmitic acid | 0.11 | 0.08 | 0.09 | 0.11 | 0.14 | 0.08 | 0.15 |
| A | Stearic acid | 0.08 | 0.06 | 0.07 | 0.08 | 0.10 | 0.06 | 0.11 |
| A | Oleic acid | 0.08 | 0.06 | 0.07 | 0.08 | 0.10 | 0.06 | 0.11 |
| A | Linoleic acid | 0.04 | 0.03 | 0.03 | 0.04 | | 0.03 | |
| A | Behenic acid | | | | | 0.12 | 0.08 | |
| A | Carnauba wax acid | 0.12 | 0.09 | 0.10 | 0.12 | | | 0.14 |
| B | Preservative | q.s. | q.s. | q.s. | q.s. | q.s. | q.s. | q.s. |
| C | Water | to | to | to | to | to | to | to |
| | | 100.0 | 100.0 | 100.0 | 100.0 | 100.0 | 100.0 | 100.0 |

| | | No. 11 | No. 12 | No. 13 | No. 14 |
|---|---|---|---|---|---|
| Phase | Example | w/w % | w/w % | w/w % | w/w % |
| A | Polyglycerol from | 6.0 | 6.0 | 6.0 | 6.0 |
| | Example 1 | | | | |
| A | Ceramide EOP | 0.001 | 0.001 | 0.001 | 0.001 |
| A | Ceramide NP | 1 | 1 | 1 | 1 |
| A | Ceramide AP | 0.5 | 0.5 | 0.5 | 0.5 |
| A | Cholesterol | 0.5 | 0.5 | 0.5 | 0.5 |
| A | Phytosphingosine | 0.5 | 0.5 | 0.5 | 0.5 |
| A | Behenic acid | 0.5 | 0.5 | 0.5 | 0.5 |
| A | Salicyloyl | | | 0.5 | |
| | Phytosphingosine | | | | |
| B | Preservative | q.s. | q.s. | q.s. | |
| C | Creatine | 1.0 | | | |
| C | Panthenol | | 5.0 | | |
| C | Nicotinamide | | | | 5.0 |
| C | Water | to 100.0 | to 100.0 | to 100.0 | to 100.0 |

| | | No 15 | No 16 | No 17 |
|---|---|---|---|---|
| Phase | Example | w/w % | w/w % | w/w % |
| A | Polyglycerol from | 6.00 | 6.00 | 6.00 |
| | Example 1 | | | |
| A | Ceramide EOP | 0.10 | 0.10 | 0.10 |
| A | Ceramide EOS | | | |
| A | Ceramide NP | 0.51 | 0.51 | 0.51 |
| A | Ceramide AP | 0.41 | 0.41 | 0.41 |
| A | Ceramide NS | 0.18 | 0.18 | 0.18 |
| A | Cholesterol | 0.47 | 0.47 | 0.47 |
| A | Potassium | 0.11 | 0.11 | 0.11 |
| | Cholesterol Sulfate | | | |
| A | Palmitic acid | 0.11 | 0.11 | 0.11 |
| A | Stearic acid | 0.08 | 0.08 | 0.08 |
| A | Oleic acid | 0.08 | 0.08 | 0.08 |
| A | Linoleic acid | 0.04 | 0.04 | 0.04 |
| A | Behenic acid | | | |
| A | Carnauba wax acid | 0.12 | 0.12 | 0.12 |
| A | Tocopherol | 1.0 | | |
| A | Tocopherol acetate | | 1.0 | |
| A | Retinol palmitate | | | 1.0 |
| B | Preservative | q.s. | q.s. | q.s. |
| C | Water | to 100.0 | to 100.0 | to 100.0 |

The formulations are prepared as follows:

Phase A and phase C are heated to 90° C. Phase A is then added to phase C with stirring. This pre-emulsion is then homogenized, for example using an Ultra Turrax. The emulsion is cooled to with gentle stirring and the preservative is added. The mixture is then further cooled to 30° C. with stirring.

### Example 4: Emulsion Stability

The formulations listed below were prepared as follows:

Polyglycerol ester, ceramides, phytosphingosine, behenic acid, glycerol monostearates, cetearyl alcohol and triethyl citrate are heated to 85° C. (phase A). The water is also heated to 85° C. Phase A is then added to the water with stirring. This pre-emulsion is then homogenized with a rotor-stator homogenizer. The emulsion is cooled to 30° C. with gentle stirring and the sodium levulinate and the potassium sorbate are added.

| | Example A (w/w %)* | Example B (w/w %) |
|---|---|---|
| Polyglycerol from | 6.0 | |
| Example 1 | | |
| Sodium Lauroyl Lactylate | | 10 |
| Ceramide EOP | 0.001 | 0.001 |
| Ceramide NP | 1 | 1 |
| Ceramide AP | 0.5 | 0.5 |
| Cholesterol | 0.5 | 0.5 |
| Phytosphingosine | 0.5 | 0.5 |
| Behenic acid | 0.5 | |
| Preservative | q.s. | q.s |
| Water | to 100.0 | to 100.0 |

| Formulation | 18 | 19 | 20 | 21 | 22 | 23 | 24 | 25 | 26 | 27 |
|---|---|---|---|---|---|---|---|---|---|---|
| Polyglycerol from Example 1 | 6.0 | | | | | | | | | |
| Polyglycerol from Example 1b | | 6.0 | | | | | | | | |
| Polyglycerol from Example 1c | | | 6.0 | | | | | | | |
| Polyglycerol from Example 1d | | | | 6.0 | | | | | | |
| Polyglyceryl-3 Palmitate¹⁾ | | | | | 6.0 | | | | | |
| Polyglyceryl-4 | | | | | | 6.0 | | | | |
| Diisostearate/Polyhydroxystearate/ | | | | | | | | | | |
| Sebacate; Caprylic/Capric Triglyceride; | | | | | | | | | | |
| Polyglyceryl-3 Oleate; Diisostearoyl | | | | | | | | | | |
| Polyglyceryl-3 Dimer Dilinoleate²⁾ | | | | | | | | | | |
| Example 5 from WO2018033259 | | | | | | | 5.0 | | | |
| Petrolatum; Ozokerite; Hydrogenated | | | | | | | 1.0 | | | |
| Castor Oil; Glyceryl Isostearate; | | | | | | | | | | |
| Polyglyceryl-3 Oleate³⁾ | | | | | | | | | | |
| Polyglyceryl-3 Stearate⁴⁾ | | | | | | | | 6.0 | | |
| Diisostearoyl Polyglyceryl-3 Dimer | | | | | | | | | 6.0 | |
| Dilinoleate⁵⁾ | | | | | | | | | | |
| Polyglyceryl-4 | | | | | | | | | | 6.0 |
| Diisostearate/Polyhydroxystearate/ | | | | | | | | | | |
| Sebacate⁶⁾ | | | | | | | | | | |

| | |
|---|---|
| Ceramide EOS | 0.001 |
| Ceramide NP | 1 |
| Ceramide AP | 0.5 |
| Cholesterol | 0.5 |
| Phytosphingosine | 0.5 |
| Behenic acid | 0.5 |
| Glycerolmonostearate | 0.5 |
| Cetearylalcohol | 0.5 |
| Triethylcitrate | 2 |
| Sodium Levulinate | 0.48 |
| Potassium Sorbate | 0.2 |
| Water | Ad 100.0 |

¹⁾ acid number = 1.7 mg KOH/g; saponification number = 160 mg KOH/g.
²⁾ ISOLAN ® 17 (Evonik)
³⁾ PROTEGIN ® W (Evonik)
⁴⁾ acid number = 1.3 mg KOH/g; saponification number = 141 mg KOH/g.
⁵⁾ ISOLAN ® PDI (Evonik)
⁶⁾ ISOLAN ® GPS (Evonik)

Formulations 18 to 21 are formed as homogeneous, stable and lamellar dispersions. Formulations 22 to 27 turned out to appear inhomogeneous already while using the rotor-stator homogenizer and phase separation occurred instantly.

1: A composition, comprising:
A) at least one ceramide,
B) at least one poly glycerol carboxylic acid ester, and
C) cholesterol and/or at least one cholesterol derivative selected from the group consisting of cholesterol sulfate, cholesterol hydrogen succinate, and 7-dehydrocholesterol,
wherein
a ratio by weight of the at least one ceramide to a sum of the cholesterol and the at least one cholesterol derivative is from 1:0.1 to 1:0.9.

2: The composition according to claim 1, wherein a ratio by weight of the at least one ceramide to the at least one polyglycerol carboxylic acid ester is from 1:1 to 1:30.

3: The composition according to claim 1, wherein the cholesterol and/or the at least one cholesterol derivative is present in a total amount of from 0.1% by weight to 3.0% by weight, wherein percentages by weight refer to a total composition.

4: The composition according to claim 1, wherein said composition comprises 20% by weight to 99% by weight of water, based on a total composition.

5: The composition according to claim 1, wherein said composition comprises component A), component B), and component C) in a total amount of 1.0% by weight to 25% by weight, based on a total composition.

6: The composition according to claim 1, wherein the at least one ceramide comprises at least two ceramides.

7: The composition according to claim 1, wherein the at least one ceramide is selected from the group consisting of ceramide NP, ceramide AP, ceramide EOP, ceramide NDS, ceramide ADS, ceramide SODS, ceramide NS, ceramide AS, ceramide EOS, ceramide NH, ceramide AH, and ceramide EOH.

8: The composition according to claim 1, wherein the at least one polyglycerol carboxylic acid ester of component B) is composed of a polyglycerol having a degree of polymerization of 2.0 to 25.

9: The composition according to claim 1, wherein the at least one polyglycerol carboxylic acid ester of component B) is composed of at least one fatty acid.

10: The composition according to claim 1, wherein, the at least one polyglycerol carboxylic acid ester of component B) is composed of a mixture of at least two selected from the group consisting of palmitic acid, stearic acid, and behenic acid,
wherein a sum of the palmitic acid, the stearic acid, and the behenic acid makes up at least 60% by weight, based on all fatty acids present in the mixture.

11: The composition according to claim 1, wherein said composition has a saponification number of 20 to 199 mg KOH/g.

12: The composition according to claim 1, wherein the composition is in a form of a cosmetic, pharmaceutical, or dermatological formulation.

13: The composition according to claim 1, wherein said composition comprises component A) component B), and component C) in a total amount of 0.01% by weight to 125% by weight, based on a total composition.

14: The composition according to claim 1, wherein said composition has a pH in a range of 4.0 to 8.0.

15: A method for increasing barrier function of skin, the method comprising:
applying the composition according to claim 1 to the skin.

16. The composition according to claim 1, wherein the ratio by weight of the at least one ceramide to the sum of the cholesterol and the at least one cholesterol derivative is from 1:0.2 to 1:0.7.

2. The composition according to claim 2, wherein the ratio by weight of the at least one ceramide to the at least one polyglycerol ca sine acid ester is from 1:2 to 1:20.

18. The composition according to claim 6, wherein the at least one ceramide comprises three ceramides.

7. The composition according to claim 7, wherein the at least one ceramide is selected from the group consisting of ceramide NP, ceramide AP, ceramide NS, ceramide EOP, and ceramide EOS.

9. The composition according to claim 9, wherein the fatty acid has 12 to 26 carbon atoms.
